(19) United States
(12) Patent Application Publication
(10) Pub. No.: US 2026/0000531 A1
(43) Pub. Date: Jan. 1, 2026

(54) DRAINABLE OSTOMY POUCH OUTLET

(71) Applicant: HOLLISTER INCORPORATED, Libertyville, IL (US)

(72) Inventors: Shane O’MALLEY, Ballina (IE); Frederik B. CHRISTIANSEN, Copenhagen (DK); Jan WOHLGEMUTH, Horsholm (DK); Kenneth NIELSEN, Fredensborg (DK)

(73) Assignee: HOLLISTER INCORPORATED, Libertyville, IL (US)

(21) Appl. No.: 19/106,353

(22) PCT Filed: Nov. 4, 2024
(86) PCT No.: PCT/US2024/054398

§ 371 Date: Feb. 25, 2025

Related U.S. Application Data
(60) Provisional application No. 63/596,437, filed on Nov. 6, 2023.

(51) Int. Cl. A61F 5/445; A61F 5/44

(52) U.S. Cl. CPC A61F 5/445; A61F 5/4407

(57) ABSTRACT

A drainable pouch includes a barrier side wall, a distal wall, an outlet, at least one closure member located on and attached to a surface of the outlet and having a kink line extending across the closure member and protruding in a direction away from the surface of the outlet. The barrier side wall and the distal side wall are joined along at least a portion of their peripheral edges to define a cavity and the outlet. The at least one closure member is configured to bend in the direction away from the surface of the outlet when opening the outlet.

### BACKGROUND

This disclosure is related to a drainable ostomy pouch outlet.

Ostomy pouches for collecting body waste are used by patients who have had surgery such as a colostomy, ileostomy, or urostomy. Ostomy pouches typically include flat, opposing side walls secured together along their edges to define a collection cavity. One of the side walls is provided with an opening to receive a stoma and means to secure the pouch to the user, such as an adhesive barrier, so that body waste discharged through the stoma is received within the cavity.

The ostomy pouch may be a closed-end pouch for single use, in which case the entire pouch is discarded after it has been substantially filled with stomal discharge. Alternatively, the ostomy pouch can be a drainable pouch with a discharge opening at its lower end, which may be closed during the collection of body waste material but may be opened for draining body waste material from the pouch after a period of use. The discharge opening of drainable pouches is typically defined at the end of a narrowed outlet portion, which is provided with closure means for maintaining the discharge opening in a sealed condition until waste material is to be drained from the pouch. However, the discharge opening may be difficult to open and close properly to ensure that a good seal is provided upon closing.

Accordingly, there is a need for an improved drainable pouch outlet.

### BRIEF SUMMARY

A drainable pouch is provided according to various embodiments.

In one aspect, the drainable pouch can include a barrier side wall, a distal wall, an outlet defining a discharge opening, and at least one closure member located on and attached to a surface of the outlet and having a kink line extending across the closure member and protruding in a direction away from the surface of the outlet. The barrier side wall and the distal wall can be joined along their peripheral edges to define a cavity and to define the outlet, the outlet being foldable upward toward the cavity. The at least one closure member may be configured to bend in the direction away from the surface of the outlet when opening the outlet.

In an embodiment, the drainable pouch can further include a securing member located on a barrier side of the outlet and a cover flap located on a distal side of the outlet for receiving and securing the securing member when the outlet is folded up.

In an embodiment, the at least one closure member can bend and open the outlet when a force beyond a threshold force is applied to outer edges of the at least one closure member.

In an embodiment, the at least one closure member can include first and second closure members. The first closure member can be located on the barrier side of the outlet, and the second closure member can be located on the distal side of the outlet. In such an embodiment, the first closure member can be located near a discharge opening of the outlet. The second closure member can be positioned to align with the first closure member and to overlie a portion of the outlet at which the first closure member is located, when the outlet is folded up.

In an embodiment, the at least one closure member may comprise a first closure member including a first kink line extending across the first closure member and protruding in a direction away from a barrier side surface of the outlet and a second closure member including a second kink line extending across the second closure member and protruding in a direction away from a distal surface of the outlet. In this embodiment, the first closure member may be attached to the barrier side wall of the outlet and the second closure member may be attached to the distal wall of the outlet and arranged vertically spaced apart from the first closure member, such that an accordion-like folds may be formed in the outlet. The first and second closure members may be configured and arranged such that the first closure member nests over the second closure member after a first folding up of the outlet. The first and second closure members may be configured to provide a tactile feedback when first closure member correctly nests over the second closure member to facilitate leak-free closure of the outlet.

In an embodiment, the first and second kink lines may be formed across the corresponding closure members in a direction perpendicular to the direction of the outlet extending from the cavity. The first and second closure members may be configured to bend away from the outlet in opposite directions.

The kink line may be formed by a fold line extending across the corresponding closure member and protruding away from the outlet or a crease extending across the corresponding closure member.

In some embodiments, each of the at least one closure member may include a cross-kink line arranged approximately in a midpoint of the closure member and configured to facilitate the closure member to bend at the midpoint in a direction away from the outlet when an inward force is applied to outer edges of the closure member to open the outlet. The cross-kink line may extend vertically in the direction of the outlet extending from the cavity. The cross-kink line may be formed by a fold line, score line, crease line, or an embossed line.

In an embodiment, the drainable pouch can further include a tab. The tab may be attached to a surface of the outlet and configured to facilitate opening the outlet to drain the cavity.

In the any of foregoing embodiments, the at least one closure member may be formed from a polymeric material.

The foregoing general description and the following detailed description are examples only and are not restrictive of the present disclosure.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1 is a barrier side view of an outlet portion of an ostomy pouch in an unfolded position, according to an embodiment.

FIG. 2 is a distal side view of the outlet portion of the ostomy pouch of FIG. 1 in an unfolded position.

FIG. 3A is a schematic cross-sectional view of the outlet portion of the ostomy pouch of FIG. 1 in an open position.

FIG. 3B is a schematic cross-sectional view of the outlet portion of the ostomy pouch of FIG. 1 folded up one time.

FIG. 3C is a schematic cross-sectional view of the outlet portion of the ostomy pouch of FIG. 1 folded up two times.

FIG. 3D is a schematic cross-sectional view of the outlet portion of the ostomy pouch of FIG. 1 folded up three times.

FIG. 3E is a schematic cross-sectional view of the outlet portion of the ostomy pouch of FIG. 1 folded up three times and secured in place with a pair of connected covers.

FIG. 4 is a distal side view of an outlet portion of an embodiment of an ostomy pouch in an unfolded position.

FIG. 5 is a bottom view of the outlet portion of the ostomy pouch of FIG. 4.

### DETAILED DESCRIPTION

While the present disclosure is susceptible of embodiment in various forms, there is shown in the drawings and will hereinafter be described presently preferred embodiments with the understanding that the present disclosure is to be considered an exemplification and is not intended to limit the disclosure to the specific embodiments illustrated. The words “a” or “an” are to be taken to include both the singular and the plural. Conversely, any reference to plural items shall, where appropriate, include the singular. The words “first,” “second,” “third,” and the like may be used in the present disclosure to describe various information, such information should not be limited to these words. These words are only used to distinguish one category of information from another. The directional words “top,” “bottom,” up,” “down,” front,” “back,” and the like are used for purposes of illustration and as such, are not limiting. Depending on the context, the word “if” as used herein may be interpreted as “when” or “upon” or “in response to determining.”

The present disclosure provides a drainable ostomy pouch that includes a drainage outlet that is easy to open and clean. The drainage outlet may include one or more closure members with a kink line that allows the drainage outlet to be correctly opened at a midpoint for cleaning and securely closed to ensure a good seal of the pouch outlet.

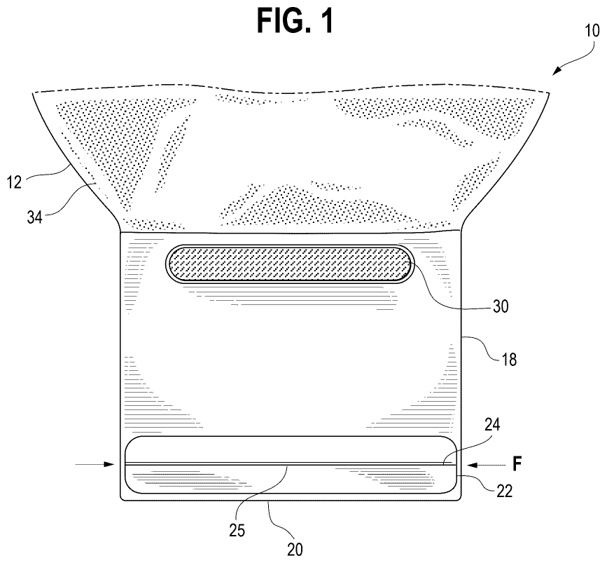

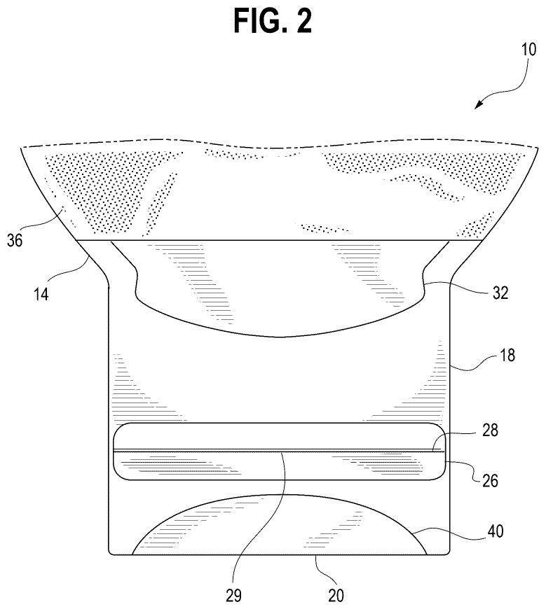

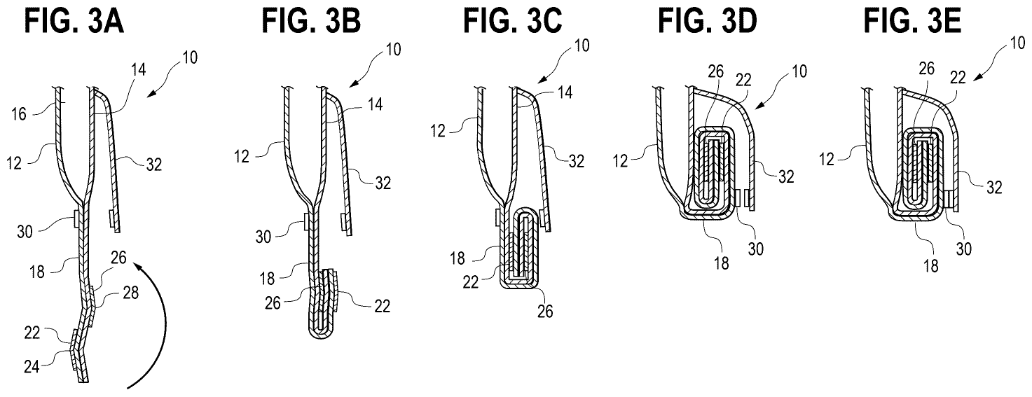

Referring now to the figures, FIGS. 1-5 a drainable pouch 10, 110 according to multiple embodiments. The drainable pouch 10, 110 can generally include a barrier or body side wall 12, a distal side wall 14, 114, a cavity 16, an outlet 18, 118, a discharge opening 20, 120, a first closure member 22 having a first kink line 24, a second closure member 26, 126 having a second kink line 28, 128, a securing member 30, a cover flap 32, 132, a barrier side cover layer 34, and a distal side cover layer 36, 136. In the embodiment shown in FIG. 2, the distal side wall 14, 114 of the outlet 18, 118 may include a distal side cutout 40, 140. The barrier side wall 12 and the distal side wall 14 can be joined along their peripheral edges to define the cavity 16 therebetween for collecting stomal discharge or effluent. The outlet 18 can terminate in a discharge opening 20 for draining the contents collected in the cavity after a period of use. The discharge opening 20 of the drainable pouch 10, 110 may be closed during use by folding outlet 18 upwardly (as shown in FIGS. 3A-3D) and securing it in the upwardly folded position as shown in FIG. 3E. The drainable pouch 10, 110, for example, may be a drainable ostomy pouch.

The closure members 22, 26 may be formed from a relatively flexible material (e.g. a polymeric material) and configured to allow the closure members 22, 26 to be bent at about their respective midpoints 25, 29 by applying an inward force F at their outer edges (FIGS. 1 and 5). As such, the closure members 22, 26 can function as living hinges, integral hinges, or other types of flexible hinges. The closure members 22, 26 may be arranged and configured to be bent in opposite directions at the midpoints 25, 29 to open the outlet 18. The kink lines 24, 28 may be configured to extend away from the outlet 18 and prevent the closure members 22, 26 from bending into the outlet 18.

In an embodiment, the closure members 22, 26 can be used to open the outlet 18 by applying pressure F on the outer edges of the closure members 22, 26, so that the closure members 22, 26 may bend or flex outwardly approximately at the midpoints 25, 29. In such an embodiment, as a threshold pressure is applied, the closure members 22, 26 may bend away from each other at their midpoints to open the outlet 18 and the discharge opening 20. The amount of force needed to bend the closure members 22, 26 and open the outlet 18, for example, the threshold pressure, can be such that the closure members 22, 26 are not inadvertently bent, for example, during an undesirable situation. The closure members 22, 26 can stay closed or in a resting position when pressure is not applied to the closure members 22, 26. The closed or resting position can prevent or reduce the risk of effluent leaking from the outlet 18 when the outlet 18 is being unfolded. When pressure is released from the sides of the closure members 22, 26, the closure members 22, 26 can return to their resting position and the outlet 18 may be closed while providing visual and tactile feedback to the user.

FIG. 1 illustrates a barrier side view of the outlet portion of the ostomy pouch 10. The first closure member 22 can be located near the end of the drainable pouch 10 on the outlet 18 and can extend across the outlet 18 in a direction perpendicular to the direction the outlet 18 extends from the drainable pouch 10. The first closure member 22 can further include the kink line 24 located along a mid-section of the first closure member 22, across the first closure member 22, and extending away from the outlet 18. The first closure member 22 can couple to the second closure member 26 to close the outlet 18 (FIGS. 3B-D). The securing member 30 can be located on the outlet 18 opposite (i.e., above) the discharge opening 20 and can extend across the outlet 18 in a direction perpendicular to the direction the outlet 18 extends from the drainable pouch 10. The securing member 30 can couple to the cover flap 32 to secure the outlet closed (FIG. 3E). The barrier cover layer 34 can be located over the barrier side wall 12 and end before the outlet 18.

FIG. 2 illustrates a distal side view of the outlet portion of the ostomy pouch 10. The second closure member 26 can be located near a mid-section of the outlet 18 to allow the first closure member 22 to fold over the second closure member 26 (FIG. 3B). The second closure member 26 can extend across the outlet 18 in a direction perpendicular to the direction the outlet 18 extends from the drainable pouch 10. The second closure member 26 can further include the kink line 28 located along a mid-section of the second closure member 26, across the second closure member 26, and extending away from the outlet 18. The cover flap 32 can be located on the ostomy pouch 10, near the outlet 18, and can extend over the outlet 18 to receive the folded-up outlet 18 and secure the outlet 18 with the secure member 30 (FIG. 3E). The discharge opening 20 can include a cutout 40, such as the illustrated arch shaped cutout, to facilitate opening the discharge opening and draining the ostomy pouch 10. The distal cover layer 36 can be located over the distal side wall 14 and end before the outlet 18. In an embodiment, the distal cover layer 36 can be located over the distal side wall 14 and can extend towards the outlet 18 up to the location of the cover 32.

In an embodiment, each of the kink lines 24, 28 may be defined by a fold line extending across the corresponding closure member 22, 26 and protruding away from the outlet 18. The closure members 22, 26 may be arranged on and attached to the opposing walls 12, 14 of the outlet 18 and configured to bend away from the outlet 18 along the kink lines 24, 28 in opposite directions to facilitate opening of the outlet 18. In the embodiments of FIGS. 1-3E, the closure members 22, 26 may be arranged vertically spaced apart on the opposite surfaces of the outlet 18, such that an accordion-like cross-sectional view can be observed in FIG. 3A. The closure members 22, 26 may be arranged such that the first closure member 22 can be stacked on top of and nested with the second closure member 26 as shown in FIG. 3B. Such a configuration of the closure members 22, 26 can facilitate correct folding up of the outlet 18 by providing a tactile feedback when the first closure member 22 correctly nests over the first closure member 26 to provide a leak-free closure of the outlet 18. In some embodiments, the kink lines 24, 28 may be defined by score lines, crease lines, embossed lines and the like that are configured to facilitate the closure members 22, 26 to bend away from the outlet 18 along the kink lines 24, 28.

FIGS. 3A-3E illustrate schematic cross-sectional views of the ostomy pouch 10. FIG. 3 A illustrates the outlet 18 in an unfolded position. The ostomy pouch 10 may be closed by folding up the outlet 18 toward the distal side wall 14 so that the closure member 22 aligns with closure member 26 and overlies that portion of the outlet 18 on which closure member 26 is located, as shown in FIG. 3B. FIGS. 3B and 3C illustrate the outlet 18 in a partially rolled-up state, and FIG. 3D illustrates the outlet 18 in a fully rolled-up state. FIG. 3E shows the outlet 18 in a fully rolled up state and in a secured position. The securing member 30 can be secured under the cover flap 32 via fasteners, such ashook-and-loop fasteners. In an embodiment, the securing member 30 and the cover flap 32 may be provided with hook-and-loop fasteners for securing the outlet 18 in the rolled up and closed position.

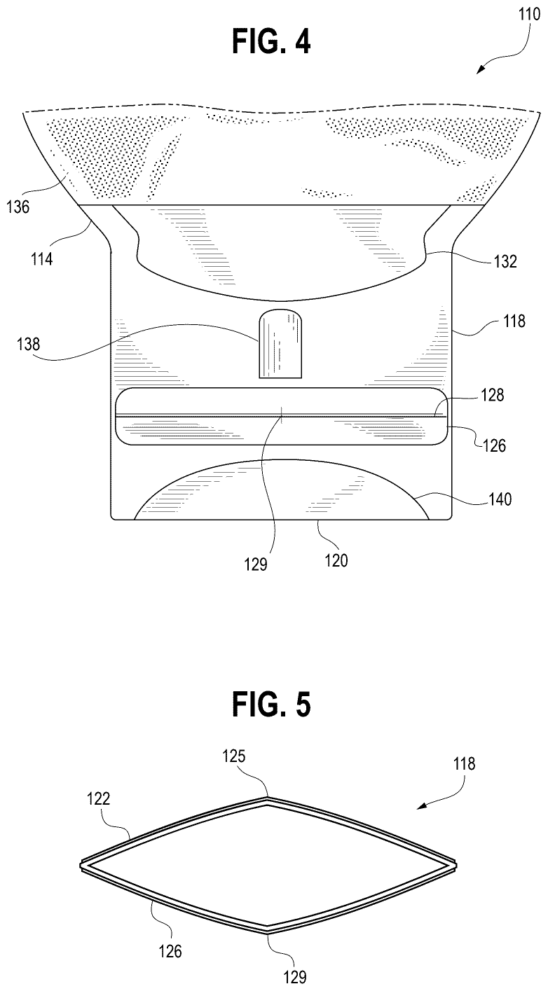

FIG. 4 illustrates a distal side view of an outlet portion of an embodiment of an ostomy pouch 110. The ostomy pouch 110 can include a tab 138 located on the outlet 118. The tab 138 can be used to facilitate opening to outlet 118 to empty the pouch 110 and/or to clean the outlet 118. For example, the tab 138 can be used to open the outlet 118 to drain effluent from the discharge opening 120. In an embodiment, the tab 138 can be located above or below the second closure member 126 (between the second closure member 126 and the outlet 118). In an embodiment, the tab 138 can include a free end oriented towards the ostomy pouch 110 (i.e., upwardly). Alternatively, the tab 138 can include a free end oriented towards a side of the outlet 118 (at an orientation perpendicular to the outlet 118). In use, a user may grab and pull the tab 138 away from the surface of the outlet 118 to facilitate opening of the outlet 118. FIG. 5 illustrates a bottom view of the outlet 118 in an open position. The closure members 122, 126 of the outlet 118 may be configured similar to the closure members 22, 26 of the outlet 18 described above, except in the embodiment of FIGS. 4 and 5, the closure members 122, 126 may be provided with cross-kink lines 125, 129 configured to facilitate the closure members 122, 126 to bend at the cross-kink lines 125, 129 when an inward force is applied to the outer edges of the closure members 122, 126 to open the outlet 118 as shown in FIG. 5. In an embodiment, each of the cross-kink lines 125, 129 may be formed by a fold line that crosses the corresponding kink-lines 128. In other embodiments, the cross-kink lines 125, 129 may be formed by score lines, crease line, embossed lines and the like.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

What is claimed is:

1. A drainable pouch, comprising:
a barrier side wall;
a distal wall;
an outlet defining a discharge opening, wherein the barrier side wall and the distal wall are joined along their peripheral edges to define a cavity and to define the outlet, the outlet being foldable upward toward the cavity; and
at least one closure member located on and attached to a surface of the outlet and having a kink line extending across the closure member and protruding in a direction away from the surface of the outlet, wherein the at least one closure member is configured to bend in the direction away from the surface of the outlet when opening the outlet.

2. The drainable pouch of claim 1, further comprising:
a securing member located on the barrier side wall of the outlet and a cover flap located on the distal side wall of the outlet for receiving and securing the securing member when the outlet is folded up.

3. The drainable pouch of claim 1, wherein the at least one closure member is configured to bend and open the outlet when a force beyond a threshold force is applied to outer edges of the at least one closure member.

4. The drainable pouch of claim 1, wherein the at least one closure member comprises first and second closure members, the first and second closure members including first and second kink lines, respectively, and wherein the first closure member is located on the barrier side wall of the outlet, and wherein the second closure member is located on the distal side wall of the outlet.

5. The drainable pouch of claim 4, wherein the first closure member is located near the discharge opening, and wherein the second closure member is positioned to align with the first closure member and to overlie a portion of the outlet at which the first closure member is located, when the outlet is folded up.

6. The drainable pouch of claim 1, wherein the at least one closure member comprises a first closure member including a first kink line extending across the first closure member and protruding in a direction away from a barrier side surface of the outlet and a second closure member including a second kink line extending across the second closure member and protruding in a direction away from a distal surface of the outlet, wherein the first closure member is attached to the barrier side wall of the outlet and the second closure member is attached to the distal wall of the outlet and arranged vertically spaced apart from the first closure member such that an accordion-like folds are formed in the outlet.

7. The drainable pouch of claim 6, wherein the first and second closure members are configured and arranged such that the first closure member nests over the second closure member after a first folding up of the outlet.

8. The drainable pouch of claim 7, wherein the first and second closure members are configured to provide a tactile feedback when first closure member correctly nests over the second closure member to facilitate leak-free closure of the outlet.

9. The drainable pouch of claim 7, wherein the first and second kink lines are formed across the corresponding closure members in a direction perpendicular to the direction of the outlet extending from the cavity.

10. The drainable pouch of claim 4, wherein the first and second closure members are configured to bend away from the outlet in opposite directions.

11. The drainable pouch of claim 1, wherein the kink line is formed by a fold line extending across the corresponding closure member and protruding away from the outlet.

12. The drainable pouch of claim 1, wherein the kink line is defined by a crease extending across the corresponding closure member.

13. The drainable pouch of claim 1, wherein each of the at least one closure member includes a cross-kink line arranged approximately in a midpoint of the closure member and configured to facilitate the closure member to bend at the midpoint in a direction away from the outlet when an inward force is applied to outer edges of the closure member to open the outlet.

14. The drainable pouch of claim 13, wherein the cross-kink line extends vertically in the direction of the outlet extending from the cavity.

15. The drainable pouch of claim 13, wherein the cross-kink line is formed by a fold line, score line, crease line, or an embossed line.

16. The drainable pouch of claim 1, wherein the at least one closure member is formed from a polymeric material.

17. The drainable pouch of any one of claims 16, further comprising:
a tab attached to a surface of the outlet and configured to facilitate opening the outlet to drain the cavity.
